(19) United States
(12) Patent Application Publication
(10) Pub. No.: US 2023/0405005 A1
(43) Pub. Date: Dec. 21, 2023

(54) TREATMENT OF GAIT DYSFUNCTION IN NEURODEGENERATIVE DISEASE

(71) Applicant: EIP Pharma, Inc., Boston, MA (US)

(72) Inventor: John Jahangir Alam, Boston, MA (US)

(73) Assignee: EIP Pharma, Inc., Boston, MA (US)

(21) Appl. No.: 18/035,543

(22) PCT Filed: Nov. 5, 2021
(86) PCT No.: PCT/US2021/058361

§ 371 Date: May 5, 2023

Related U.S. Application Data
(60) Provisional application No. 63/110,922, filed on Nov. 6, 2020.

(51) Int. Cl. A61K 31/519; A61P 25/28

(52) U.S. Cl. CPC A61K 31/519; A61P 25/28

(57) ABSTRACT

The present invention provides methods and compositions for treatment of gait dysfunction in subjects with neurodegenerative disease of the cholinergic forebrain. In some embodiments, methods are provided for treatment of gait dysfunction related to an alpha synuclein disease, such as Parkinson's disease (PD) or Dementia with Lewy Bodies (DLB).

### CROSS-REFERENCE TO RELATED APPLICATIONS

This application claims priority to U.S. Provisional Application No. 63/110,922 filed Nov. 6, 2020, the entire content of which is incorporated herein by reference. herein.

### BACKGROUND

Gait dysfunction is a debilitating symptom of neurodegenerative diseases, such as Parkinson's disease. Gait dysfunction is often refractive to dopaminergic therapies that have been employed for treatment of PD.

### SUMMARY

Impairment in gait and balance is a feature of neurodegenerative diseases that affect forebrain cholinergic neurons, such as Parkinson's disease (PD) or Dementia with Lewy Bodies. For example, patients with PD typically suffer from postural instability, reduced gait speed, reduced stride length, slower turns, freezing of gait, and falls. Gait impairment and freezing of gait affect approximately 75% of individuals with advanced PD.

The present disclosure encompasses the discovery that selective p38α mitogen activated protein kinase (MAPK) inhibitors can be used to inhibit or reverse effects or symptoms of Parkinson's disease (e.g., gait dysfunction). In particular, it has been found that administration of the p38α MAPK inhibitor neflamapimod can improve motor symptoms in human subjects suffering from neurodegenerative diseases of the forebrain cholinergic system.

Provided herein are methods of treating gait dysfunction. In some embodiments, a method is provided for treatment of gait dysfunction in a subject afflicted with forebrain cholinergic neuron degeneration, the method comprising administering neflamapimod to the subject.

In some embodiments, forebrain cholinergic neuron degeneration comprises degeneration of the nucleus basalis of Meynert (NBM).

In some embodiments, provided herein is a method of treating a subject having an alpha synuclein disease, the method comprising administering neflamapimod to the subject.

In some embodiments, the alpha synuclein disease is Parkinson's disease (PD).

In some embodiments, the alpha synuclein disease is Dementia with Lewy Bodies (DLB).

In some embodiments, provided herein is a method of treating a subject having Parkinson's disease, the method comprising administering neflamapimod to the subject. In some embodiments, the neflamapimod is administered to alleviate bradykinesia, rigidity, resting tremor, postural instability, fall risk, or gait dysfunction.

In some embodiments, provided herein is a method of administering neflamapimod to a subject having Parkinson's disease to alleviate gait dysfunction.

In some embodiments, the subject suffers from continuous gait dysfunction. In some embodiments, the subject suffers from episodic gait dysfunction.

In some embodiments, provided herein is the use of neflamapimod in the manufacture of a medicament for treatment of PD.

In some embodiments, provided herein is a pharmaceutical composition comprising neflamapimod for treatment of PD. In some embodiments, provided herein is the use of neflamapimod in a method of treating PD.

In some embodiments, the daily amount of neflamapimod administered is equivalent to a dose of 40 mg (BID). In some embodiments, the daily amount of neflamapimod administered is equivalent to a dose of 40 mg (TID). In some embodiments, methods herein comprise administering to a subject neflamapimod at a dose of 40 (BID). In some embodiments, methods herein comprise administering to a subject neflamapimod at a dose of 40 (TID).

### BRIEF DESCRIPTION OF THE DRAWINGS

FIG. 1A illustrates the Timed Up and Go (TUG) test, wherein the time it takes a subject to stand up from a chair, walk three meters, turn around and return to the chair is measured.

FIG. 1B shows the effect of a selective p38α MAPK inhibitor, neflamapimod, as tested using TUG tests after treating human subjects over the course of 16 weeks.

FIG. 2 shows effect of neflamapimod treatment compared to placebo on TUG test results in human subjects over the course of 16 weeks. p=0.044 for placebo vs. NFMD, Mixed Model for Repeated Measures (MMRM).

FIG. 3 shows effect of neflamapimod treatment 40 mg TID compared to placebo on TUG test results in human subjects over the course of 16 weeks. p=0.024 for placebo TID vs. NFMD, Mixed Model for Repeated Measures (MMRM).

FIG. 4 shows effect of neflamapimod treatment compared to placebo on Personal Care Domain of Clinical Dementia Rating Scale Sum of Boxes (CDR-SB) test results in human subjects over the course of 16 weeks. p=0.02 for placebo TID vs. NFMD, Mixed Model for Repeated Measures (MMRM).

FIG. 5 shows effect of neflamapimod treatment compared to placebo on beta functional connectivity on EEG in human subjects. “40 mg” refers to combined neflamapimod patients, including 40 mg BID and 40 mg TID.

### DEFINITIONS

Carrier: The term “carrier” refers to any chemical entity that can be incorporated into a composition containing an active agent (e.g., a p38α MAPK inhibitor) without significantly interfering with the stability and/or activity of the agent (e.g., with a biological activity of the agent). In certain embodiments, the term “carrier” refers to a pharmaceutically acceptable carrier.

Formulation: The term “formulation” as used herein refers to a composition that includes at least one active agent (e.g., p38α MAPK inhibitor) together with one or more carriers, excipients or other pharmaceutical additives for administration to a patient. In general, particular carriers, excipients and/or other pharmaceutical additives are selected in accordance with knowledge in the art to achieve a desired stability, release, distribution and/or activity of active agent(s) and which are appropriate for the particular route of administration.

Pharmaceutically acceptable carrier, adjuvant, or vehicle: The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Therapeutically effective amount and effective amount: As used herein, and unless otherwise specified, the terms “therapeutically effective amount” and “effective amount” of an agent refer to an amount sufficient to provide a therapeutic benefit in the treatment, prevention and/or management of a disease, disorder, or condition, e.g., to delay onset of or minimize (e.g., reduce the incidence and/or magnitude of) one or more symptoms associated with the disease, disorder or condition to be treated. In some embodiments, a composition may be said to contain a “therapeutically effective amount” of an agent if it contains an amount that is effective when administered as a single dose within the context of a therapeutic regimen. In some embodiments, a therapeutically effective amount is an amount that, when administered as part of a dosing regimen, is statistically likely to delay onset of or minimize (reduce the incidence and/or magnitude of) one or more symptoms or side effects of a disease, disorder or condition.

Treat or Treating: The terms “treat” or “treating,” as used herein, refer to partially or completely alleviating, inhibiting, delaying onset of, reducing the incidence of, yielding prophylaxis of, ameliorating and/or relieving or reversing a disorder, disease, or condition, or one or more symptoms or manifestations of the disorder, disease or condition.

Unit Dose: The expression “unit dose” as used herein refers to a physically discrete unit of a formulation appropriate for a subject to be treated (e.g., for a single dose); each unit containing a predetermined quantity of an active agent selected to produce a desired therapeutic effect when administered according to a therapeutic regimen (it being understood that multiple doses may be required to achieve a desired or optimum effect), optionally together with a pharmaceutically acceptable carrier, which may be provided in a predetermined amount. The unit dose may be, for example, a volume of liquid (e.g., an acceptable carrier) containing a predetermined quantity of one or more therapeutic agents, a predetermined amount of one or more therapeutic agents in solid form (e.g., a tablet or capsule), a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included. It will be understood, however, that the total daily usage of a formulation of the present invention will be decided by the attending physician within the scope of sound medical judgment. The specific effective dose level for any particular subject may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. In some embodiments, a unit dose of a p38 MAPKα inhibitor is about 1 mg, 3 mg, 5 mg, 10 mg, 15 mg, 20 mg, 25 mg, 30 mg, 35 mg, 40 mg, 45 mg, 50 mg, 60 mg, 80 mg, 100 mg, 125 mg, or 250 mg.

### DETAILED DESCRIPTION OF CERTAIN EMBODIMENTS

The present disclosure provides, among other things, compositions and methods for treating motor symptoms, e.g., gait dysfunction, in subjects with Parkinson's disease or DLB or associated symptoms thereof, by administering a composition comprising a selective p38α MAPK inhibitor. In some embodiments, the p38α MAP inhibitor is neflamapimod.

In some embodiments, the disclosure provides compositions and methods for treating subjects susceptible to or at risk of developing Parkinson's disease (e.g., people with family history of Parkinson's disease, people who have suffered head trauma, or people exposed to certain chemicals (e.g., pesticides)) or subjects susceptible to or at risk of developing DLB (e.g., people with family history of DLB, people who have suffered head trauma, or people exposed to certain chemicals (e.g., pesticides)).

Various aspects of the disclosure are described in detail in the following sections. The use of sections is not meant to limit the disclosure. Each section can apply to any aspect of the disclosure.

### Gait Dysfunction

Gait dysfunction is a symptom in certain patients with alpha synuclein associated neurodegenerative disease, such Parkinson's disease (PD). Typically, such patients walk slowly with shuffling and dragging steps, diminished arm swing and flexed forward posture.

Higher-order aspects of gait control, such as gate variability, are impaired by loss or dysfunction of forebrain cholinergic neurons. Cortical cholinergic degeneration in the Ch4 brain area, which includes the nucleus basalis of Meynert (NBM) is associated with impairment in gait and balance, such as in PD.

### Parkinson's Disease

While the predominant pathology in Parkinson's disease (PD) is loss of dopaminergic neurons in the basal ganglia, PD is also associated with degeneration of the cholinergic basal forebrain (cholinergic nuclei 1-4) and ultimately degeneration of cholinergic neurons in the upper brainstem. Cholinergic nucleus 4 (Ch4), which includes the nucleus basalis of Meynert (NBM), has widespread cholinergic projections to the neocortex, while cholinergic nuclei 1,2,3 (Ch123) mainly project to the olfactory bulb and hippocampus. Cholinergic denervation in the cortex arising from degeneration of Ch4 is associated with impairment in gait and balance in PD, particularly reduced gait speed.

Parkinson's disease involves motor and/or nonmotor symptoms. Some non-limiting examples of motor symptoms include bradykinesia, rigidity, resting tremor, postural instability, imbalance, loss of automatic movements, speech changes, writing changes, change in facial expression (e.g., masked face), fall risk, and/or gait dysfunction. Some non-limiting examples of nonmotor symptoms include depression, fatigue, and sleep disorders. In some embodiments, subjects with Parkinson's disease suffer from autonomic problems. The Diagnostic and Statistical Manual of Mental Disorders provides criteria for identifying a subject or patient suffering from Parkinson's disease.

In some embodiments, methods disclosed herein can be used to treat a Parkinsonian disease/disorder or symptoms (e.g., motor symptoms) thereof.

In some embodiments, methods disclosed herein can be used to alleviate gait dysfunction associated with neurodegenerative disease, such as PD or DLB. Gait dysfunction, and effective treatment thereof, can be assessed using known tests, such as the Timed Up and Go (TUG) test, Fast Walking Speed (FWS) test, or stepping-in-place (SIP) task, which is a metric of gait impairment and freezing. Alleviation of gait dysfunction can be assessed by an improvement in scores from baseline (i.e., in the absence of neflamapimod) on any one of the foregoing tests. In some embodiments, alleviation of gait dysfunction is measured by improved scores in the TUG test.

### P38 MAPK

Many extracellular stimuli, including pro-inflammatory cytokines and other inflammatory mediators, elicit specific cellular responses through the activation of mitogen-activated protein kinase (MAPK) signaling pathways. MAPKs are proline-targeted serine-threonine kinases that transduce environmental stimuli to the nucleus. Once activated, MAPKs activate other kinases or nuclear proteins through phosphorylation, including potential transcription factors and substrates. The four isoforms (α, β, δ, and γ) of p38 MAP kinase comprise one specific family of MAPKs in mammals that mediate responses to cellular stresses and inflammatory signals.

Pharmacological inhibitors of p38 MAPK have been developed as potential therapeutics for a variety of disorders. These include compounds that inhibit α, β, γ, δ isoforms of p38 MAPK (pan inhibitors), such as SB239063, compounds that inhibit both α and β isoforms such as RWJ67657, and compounds that selectively inhibit the α isoform, such as neflamapimod (VX-745) and BMS582949 (for review, see Shahin et al., (2017) Future Sci OA, 3(4) FSO204).

In some experimental paradigms, the pharmacological effects of pan inhibitors are distinguishable from those of isoform selective inhibitors. For example, in hippocampal cell culture, the pan p38 MAPK inhibitor SB239063 was found to be ineffective against amyloid β-derived diffusible ligand (ADDL) induced synaptotoxicity, whereas neflamapimod, a p38α selective MAPK inhibitor, showed positive effects (see Fang et al. PLoS (2018), 1-32, Amin et al., “Role of p38α MAP kinase in amyloid-β derived diffusible ligand (ADDL) induced dendritic spine loss in hippocampal neurons,” Alzheimer's Association International Conference, July 2019). However, in addition to inhibition of p38 MAPK, SB239063 has also been reported to inhibit casein kinase isoforms CKIδ and CKIε (Verkaar et al. (2011) Chem. & Biol., 18:485-494).

Some studies have shown an involvement of p38 MAPK in the pathobiology of Parkinson's disease (Obergasteiger et al. Molecular Neurodegeneration (2018) 13:40; He et al., Translational Neuroscience 9, 2018, 147-153; and Chen et al. Cell Death and Disease (2018) 9:700). However, other studies suggest that α-synuclein interferes with p38γ, but not p38α, in the mechanisms of synaptic dysfunction in Parkinson's disease (He et al., Front. Neurosci. March 2020, 14: article 286).

### Neflamapimod

Neflamapimod is a small-molecule selective inhibitor of the alpha isoform of p38 MAPK. Neflamapimod, also known as VX-745, has a chemical name of 5-(2,6-dichlorophenyl)-2-(2,4-difluorophenylthio)-6H-pyrimido[1,6-b]pyridazin-6-one.

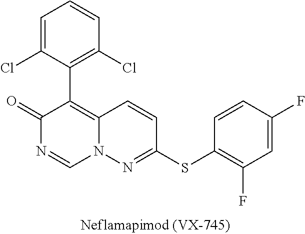 [SMILES: O=c1ncn2nc(Sc3ccc(F)cc3F)ccc2c1-c1c(Cl)cccc1Cl]

### Pharmaceutical Compositions

In some embodiments, a provided method comprises administering to a patient a pharmaceutical composition comprising a p38α MAPK inhibitor, such as neflamapimod, together with one or more therapeutic agents and a pharmaceutically acceptable carrier or vehicle. In some embodiments, the present invention provides a pharmaceutical composition comprising a dose of p38α MAPK inhibitor together with one or more therapeutic agents and a pharmaceutically acceptable carrier or vehicle, wherein the dose of p38α MAPK inhibitor results in an average blood concentration of from about 1 ng/mL to about 15 ng/mL, from about 1 ng/mL to about 10 ng/mL, from about 5 ng/mL to about 15 ng/mL, or from about 5 ng/mL to about 10 ng/mL.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Dosing

In some embodiments, compositions are administered in a therapeutically effective amount and/or according to a dosing regimen that is correlated with a particular desired outcome (e.g., with treating or reducing risk for disease).

In some embodiments, provided compositions are administered in a therapeutically effective amount and/or according to a dosing regimen that is correlated with a particular desired outcome (e.g., reduction in symptoms, such as gait dysfunction, of Parkinson's disease or DLB, etc.).

In some embodiments, a therapeutically effective amount of neflamapimod is equivalent to a dose of 40 mg administered BID. In some embodiments, a therapeutically effective amount of neflamapimod is equivalent to a dose of 40 mg administered TID.

Alternatively or additionally, in some embodiments, an appropriate dose or amount is determined through use of one or more in vitro or in vivo assays to help identify desirable or optimal dosage ranges or amounts to be administered.

In various embodiments, provided compositions are administered at a therapeutically effective amount. As used herein, the term “therapeutically effective amount” or “therapeutically effective dosage amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition).

In some embodiments, a composition is provided as a pharmaceutical formulation. In some embodiments, a pharmaceutical formulation is or comprises a unit dose amount for administration in accordance with a dosing regimen correlated with achievement of disease reduction in symptoms of prion disease, arrest or decrease in rate of decline of function due to prion disease.

In some embodiments, a formulation comprising provided compositions as described herein is administered as a single dose. In some embodiments, a formulation comprising provided compositions as described herein is administered as two doses. In some embodiments, a formulation comprising provided compositions as described herein is administered as three doses. In some embodiments, a formulation comprising provided compositions as described herein is administered at regular intervals. Administration at an “interval,” as used herein, indicates that the therapeutically effective amount is administered periodically (as distinguished from a one-time dose). The interval can be determined by standard clinical techniques. In some embodiments, a formulation comprising provided compositions as described herein is administered twice weekly, thrice weekly, every other day, daily, twice daily, thrice daily, or every eight hours.

In some embodiments, a formulation comprising provided compositions as described herein is administered once daily. In some embodiments, a formulation comprising provided compositions as described herein is administered twice daily. In some embodiments, the twice daily administering occurs from about 9 to 15 hours apart. In some embodiments the twice daily administering occurs about 12 hours apart. In some embodiments, a formulation comprising provided compositions as described herein is administered thrice daily. In some embodiments, the three times daily administering occurs from about 4 to 8 hours apart. In some embodiments, the thrice daily administering occurs from about 6 to 12 hours apart. In some embodiments the thrice daily administering occurs about 8 hours apart. In some embodiments, a formulation comprising from about 20 mg to about 250 mg of neflamapimod is administered twice daily. In some embodiments, a formulation comprising from about 20 mg to about 250 mg of neflamapimod is administered thrice daily. In some embodiments, a formulation comprising from about 40 mg to about 250 mg of neflamapimod is administered twice daily. In some embodiments, a formulation comprising from about 40 mg to about 250 mg of neflamapimod is administered thrice daily. In some embodiments, the administering occurs when the patient is in a fed state. In some embodiments, the administering occurs within 30 to 60 minutes after the subject has consumed food. In some embodiments, the administering occurs when the patient is in a fasted state. The administration interval for a single individual need not be a fixed interval, but can be varied over time, depending on the needs of the individual.

In some embodiments, a formulation comprising provided compositions as described herein is administered at regular intervals. In some embodiments, a formulation comprising provided compositions as described herein is administered at regular intervals for a defined period. In some embodiments, a formulation comprising provided compositions as described herein is administered at regular intervals for 2 years, 1 year, 11 months, 10 months, 9 months, 8 months, 7 months, 6 months, 5 months, 4 months, 3 months, 2 months, a month, 3 weeks, 2, weeks, a week, 6 days, 5 days, 4 days, 3 days, 2 days or a day. In some embodiments, a formulation comprising provided compositions as described herein is administered at regular intervals for 16 weeks.

In some embodiments, a p38α inhibitor is administered to a subject to provide acute treatment of Parkinson's disease or symptoms thereof (e.g., motor symptoms) or DLB or symptoms thereof (e.g., motor symptoms). In some embodiments, a p38α inhibitor is administered to a subject to improve gait dysfunction. In some embodiments, the subject that is administered neflamapimod is also receiving cholinesterase inhibitor therapy.

### EXEMPLIFICATION

The following examples are provided for illustrative purposes and are not intended to limit the scope of the invention.

### Example 1

This example demonstrates that neflamapimod is effective to treat motor symptoms in humans having an alpha synuclein disease that leads to forebrain cholinergic degeneration, such as PD or DLB.

### Study Design

A double-blind placebo-controlled, 16-week, treatment with neflamapimod was conducted in parallel groups at 22 centers in the US and 2 centers in the Netherlands.

Inclusion criteria included Mild-to-moderate (MMSE 15-28) probable Dementia with Lewy Bodies by consensus criteria (McKeith et al, Neurology, 2017; 89:88-100), a positive reading of DaTscan™, and have to have been currently receiving cholinesterase inhibitor therapy (>3 months and stable dose >6 weeks). If DaTscan™ was negative, subjects could also be enrolled with history of polysomnography-confirmed REM sleep disorder (6 study participants so enrolled). The study was randomized 1:1 to 40 mg neflamapimod or matching-placebo capsules. Dosing regimen was based on weight: subjects weighing <80 kg received capsules twice-daily (BID) and those weighing ≥80 kg received capsules three-times-a-day (TID).

Table 1 provides baseline characteristics of the patient or subject population.

TABLE 1

| | Placebo BID | NEM BID | Placebo TID | NEM BID |
|---|---|---|---|---|
| | (N = 18) | (N = 26) | (N = 27) | (N = 20) |
| Age (yrs) | 75 (7.6) | 74 (6.5) | 70 (5.7) | 72 (6.6) |
| Male | 72% | 73% | 96% | 95% |
| CDR ≤0.5/1.0/≥2.0 | 17%/67%/17% | 31%/50%/19% | 55%/37%/7% | 40%/60%/0% |
| CDR Sum of Boxes | 6.3 (3.2) | 5.7 (2.9) | 4.3 (2.3) | 4.7 (1.8) |
| MMSE | 22.5 (3.3) | 22.4 (3.7) | 23.6 (3.3) | 23.6 (3.7) |

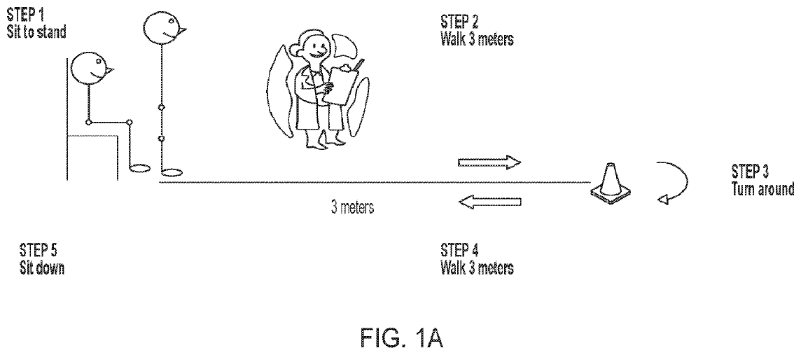

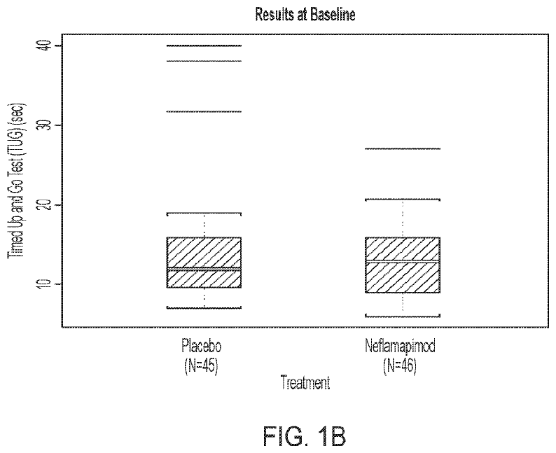

Secondary clinical endpoints included the following. (a) International Shopping List Test (ISLT); (b) Timed Up and Go Test (TUG), Ten item Neuropsychiatric Inventory (NPI-10); and (c) Clinical Dementia Rating scale Sum-of-Boxes (CDR-SB). The TUG test is useful to measure risk of fall in patients with or at risk of suffering from Parkinson's disease (see e.g., Arch Phys Med Rehabil. 2013 July; 94(7): 1300-1305). FIG. 1A shows provides a description of the test. FIG. 1B shows the effect of a p38 MAPK inhibitor, neflamapimod, as tested using TUG tests after treating human subjects as described above over the course of 16 weeks.

### Results

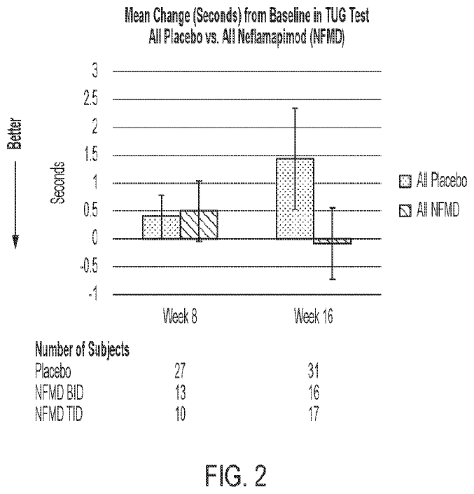

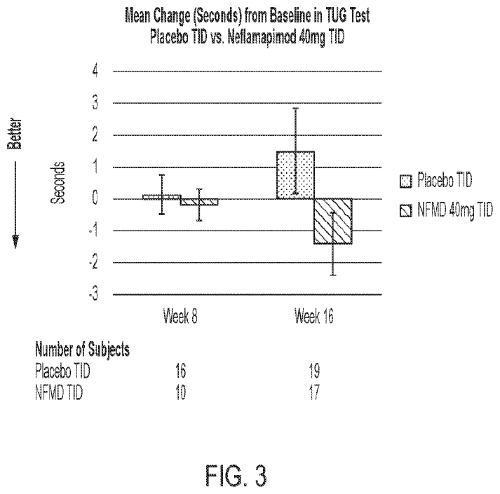

FIG. 2 and FIG. 3 show results for TUG tests at 8 and 16 weeks after commencement of the neflamapimod (or placebo) treatment. In a comparison of all placebo vs. all neflamapimod (i.e., both 40 mg BID and 40 mg TID) treatment, neflamapimod was found to significantly improve outcome (FIG. 2). In a comparison of placebo TID vs. neflamapimod 40 mg TID treatment, neflamapimod was found to significantly improve outcome (FIG. 3).

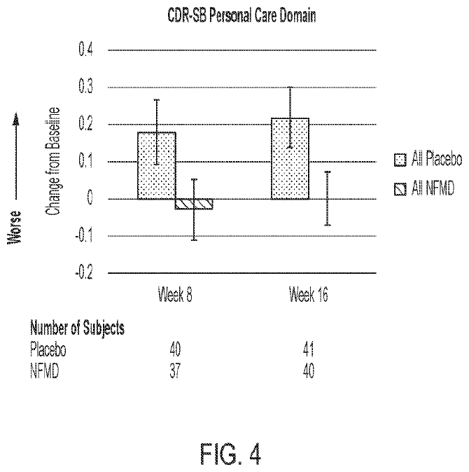

FIG. 4 shows neflamapimod also had a significant positive outcome compared to placebo treatment in personal care domain of the CDR sum of boxes, which is the domain within CDR-SB that is most dependent on motor function.

### Example 2

This example demonstrates the ability of neflamapimod to directly impact functional connectivity in regions of the brain responsible for complex motor control. These regions, in particular the frontal cortex, receive cholinergic input, including projections from the nucleus basalis of Meynert. Dysfunction of the cholinergic system leads to impaired cortical functional connectivity, which is disruptive to the performance of complex motor functions such as gait. The ability of neflamapimod to improve functional connectivity, as measured by EEG in the experiments described herein, provides further support for the efficacy of neflamapimod to alleviate gait dysfunction.

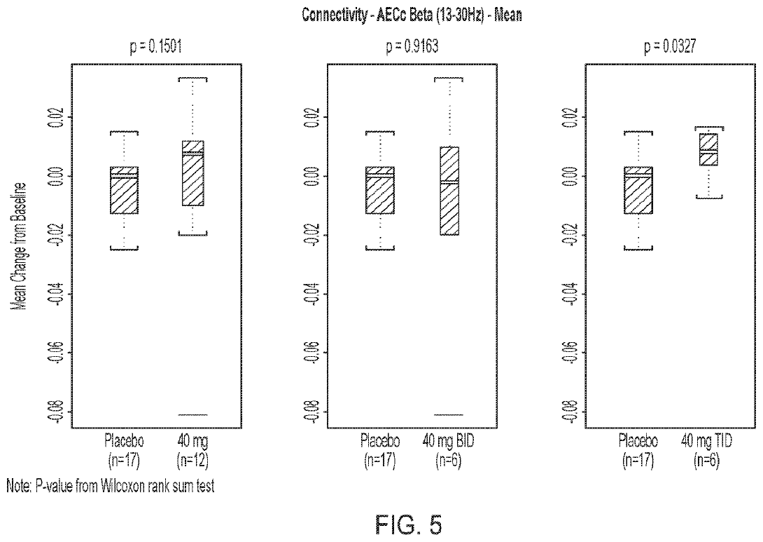

FIG. 5 shows effect of neflamapimod treatment on beta functional connectivity using EEG. Functional connectivity analysis, specifically corrected Amplitude Envelope Correlation (AECc), measures interregional communication or so-called ‘functional connectivity’ between different brain regions. In the functional connectivity analysis, a positive neflamapimod treatment effect on AECc in the beta band (13-30 Hz) was identified. Mean AECc beta was increased with neflamapimod TID versus all placebo (p=0.033) and versus placebo TID (p=0.01). The effect was most prominent in the frontal region (p=0.009 for placebo TID versus neflamapimod TID), but statistically significant difference for that comparison also seen in the temporal (p=0.036) and parietal (p=0.036) regions. AECc in each of the other bands was stable or slightly improved with no treatment group differences.

In summary, the clinical data provided herein demonstrate that treatment with neflamapimod results in improvement of motor function and alleviates symptoms associated with PD such as gait dysfunction.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A method of treating gait dysfunction in a subject afflicted with forebrain cholinergic neuron degeneration, the method comprising administering neflamapimod to the subject.

2. The method of claim 1, wherein the forebrain cholinergic neuron degeneration comprises degeneration of the nucleus basalis of Meynert (NBM).

3. The method of claim 1, wherein the subject has an alpha synuclein disease.

4. The method of claim 1, wherein the subject has Parkinson's disease.

5. The method of claim 1, wherein the subject has Dementia with Lewy Bodies (DLB).

6. A method of treating a subject having Parkinson's disease, the method comprising administering neflamapimod to the subject.

7. The method of claim 6, wherein the neflamapimod is administered to alleviate bradykinesia, rigidity, resting tremor, postural instability, fall risk, or gait dysfunction.

8. The method of claim 6, wherein the neflamapimod is administered to alleviate gait dysfunction.

9. The method of claim 8, wherein the gait dysfunction is continuous.

10. The method of claim 8, wherein the gait dysfunction is episodic.
